(19) United States
(12) Patent Application Publication
(10) Pub. No.: US 2012/0130369 A1
(43) Pub. Date: May 24, 2012

(54) REVERSIBLE ELECTROPORATION DEVICE FOR INDUCING CELL APOPTOSIS

(76) Inventors: Ruggero Cadossi, Carpi (IT); Donata Marazzi, Carpi (IT); Francesca De Terlizzi, Mogliano Veneto (IT); Mattia Ronchetti, Carpi (IT)

(21) Appl. No.: 13/318,684

(22) PCT Filed: May 3, 2010
(86) PCT No.: PCT/IB10/01002

§ 371 Date: Feb. 9, 2012

(30) Foreign Application Priority Data
May 4, 2009 (IT) TO2009A000353

(51) Int. Cl. A61B 18/00

(52) U.S. Cl. 606/41

(57) ABSTRACT

A reversible electroporation device in which an electric pulse generator generates a sequence of electric pulses delivered to electrodes coupled to a tissue in which a process of reversible electroporation that generates cell apoptosis is to be performed. The device is configured to control the amplitude of the pulses and the number of the pulses delivered to the electrodes so that the amount of energy per weight unit (absorbed dose) applied to the tissue is in a range between a first lower limit value of about 3000 J/kg and a second upper limit value of about 4500 J/kg.

### TECHNICAL FIELD

The present invention relates to a reversible electroporation device and method adapted to induce cell apoptosis.

### BACKGROUND ART

As is known, in many surgical and ablative treatments (e.g. directed to eliminate malignant or benign tumours), it is important to eliminate a specific pathological tissue without causing damage, or limiting it at minimum, to surrounding healthy tissues which do not need to be removed.

In this field, irreversible electroporation treatments have recently been developed which are adapted to eliminate specific portions of target cell tissue leaving the surrounding portions unaltered.

As is known, electroporation treatments provide for the application of electric pulses to a tissue by means of the use of electrodes applied to the tissue; the generated electric field induces the formation of pores in the cell membrane causing a variation thereof that promotes the flow of organic/inorganic substances (e.g. DNA or drugs) from the outside to the inside of the cell.

These electroporation treatments may be controlled on the basis of parameters (voltage, waveform, duty cycle, application time, number of applied pulses, etc.) of the electric pulses; by varying such parameters a reversible or irreversible electroporation may be obtained.

In the first case, the pores of the cell membrane close upon the interruption of the pulses and cells remain viable. In the second case, the high electric field applied to the cells produces pores having a size such that the integrity of the cell membrane may not be restored and determines cell lysis. Limited thermal effects occur at the surface of the electrodes.

Document WO 2005065284 in the name of DAVALOS, Rafael, and RUBINSKY, Boris discloses the limits of electroporation processes in detail.

In particular, it should be noted (FIG. 1 of the above mentioned PCT patent application is attached hereto for this purpose) that by controlling the intensity of the applied field (Y axis) and the amplitude of the pulses (X axis), the result obtained may be:
none;
a reversible electroporation (area shown by the dotted line); or
an irreversible electroporation that causes the lysis of the cells.

On the basis of what is shown in the above mentioned document, an irreversible electroporation is obtained by applying pulses having a duration of more than at least 100 microseconds and having a high enough amplitude. This causes an irreversible damage to the cell membrane causing cell death. The formation of reversible pores should not lead to cell lysis or cell death. Cell lysis occurs when electric pulses have a high amplitude and sufficient duration. This is shown in the figure by the area above the dotted line (upper curve). The application of such pulses results in a high electron flow that causes a diffused inflammatory condition of the tissue.

### DISCLOSURE OF INVENTION

It is an object of the present invention to provide a reversible electroporation device and method, which induces cell destruction by a mechanism other that cell lysis, thus producing a reduced inflammation.

This object is achieved by the present invention as it relates to a reversible electroporation device adapted to induce cell apoptosis, comprising an electric pulse generator adapted to output a sequence of electric pulses having a predetermined waveform delivered to electrodes coupled to a tissue in which a process of reversible electroporation is to be performed, characterized in that the device is configured to control the amplitude of the pulses and the number of pulses delivered to the electrodes so as to supply an amount of energy per weight unit (absorbed dose) in a range between a first lower limit value of about 3000 J/kg and a second upper limit value of about 4500 J/kg.

The present invention also relates to a reversible electroporation method adapted to induce cell apoptosis, wherein there is provided the step of delivering a sequence of electric pulses having a predetermined waveform to electrodes coupled to a tissue in which a process of reversible electroporation is to be performed,

characterised by comprising the step of controlling the amplitude of the pulses and the number of pulses delivered to the electrodes so as to supply an amount of energy per weight unit (absorbed dose) in a range between a first lower limit value of about 3000 J/kg and a second upper limit value of about 4500 J/kg.

### BRIEF DESCRIPTION OF THE DRAWINGS

The invention will now be described with reference to the accompanying figures, which set forth a non-limitative embodiment thereof, in which:

FIG. 1 shows the known art;

FIG. 2 diagrammatically shows an electroporation device made according to the present invention;

FIG. 3 shows a diagram of the number of pulses as a function of the absorbed dose for different voltages highlighting the dose range between 3000 J/kg and 4500 J/kg;

FIG. 4 shows a diagram of the number of pulses as a function of the value of the electric field applied to the electrodes. Each curve refers to a different value of absorbed dose in J/kg;

FIG. 5A is a photograph of the introduction of 2 electrodes in the distal femur of a rabbit;

FIG. 5B is a diagrammatic view of the arrangement of a pair of electrodes;

FIG. 5C is a diagrammatic view of the arrangement of four electrodes;

FIG. 6A is a fluorescence image of a tissue (rabbit distal femur) subjected to electroporation with 1000 V/cm, 180 pulses and absorbed dose equivalent to 1700 J/kg;

FIG. 6B is a haematoxylin-eosin histological image with of a tissue (rabbit distal femur) subjected to electroporation with 1000 V/cm, 180 pulses and absorbed dose equivalent to 1700 J/kg;

FIG. 7A is a fluorescence image of a tissue (rabbit distal femur) subjected to electroporation with 1000 V/cm, 480 pulses and absorbed dose equivalent to 4542 J/kg;

FIG. 7B is a haematoxylin-eosin histological image of a tissue (rabbit distal femur) subjected to electroporation with 1000 V/cm, 480 pulses and absorbed dose equivalent to 4542 J/kg;

FIG. 8A is a haematoxylin-eosin histological image of a tissue subjected to electroporation with 1300 V/cm, 160 pulses and absorbed dose equivalent to 2550 J/kg with a 4 electrode configuration in the rabbit distal femur;

FIG. 8B is a haematoxylin-eosin histological image of a tissue subjected to electroporation with 1300 V/cm, 360 pulses and absorbed dose equivalent to 5749 J/kg with a 4 electrode configuration in the rabbit distal femur;

FIG. 9A is a toluidine blue histological image of a tissue (rabbit distal femur) subjected to electroporation with 1500 V/cm, 120 pulses and absorbed dose equivalent to 2890 J/kg;

FIG. 9B is a toluidine blue histological image of a tissue (rabbit distal femur) subjected to electroporation with 1500 V/cm, 150 pulses and absorbed dose equivalent to 3598 J/kg;

FIG. 10A is a histological image of a tissue (sheep vertebra) subjected to electroporation with 1750 V/cm, 60 pulses and absorbed dose equivalent to 1700 J/kg; and

FIG. 10B is a histological image of a tissue (sheep vertebra) subjected to electroporation with 1750 V/cm, 160 pulses and absorbed dose equivalent to 4633 J/kg.

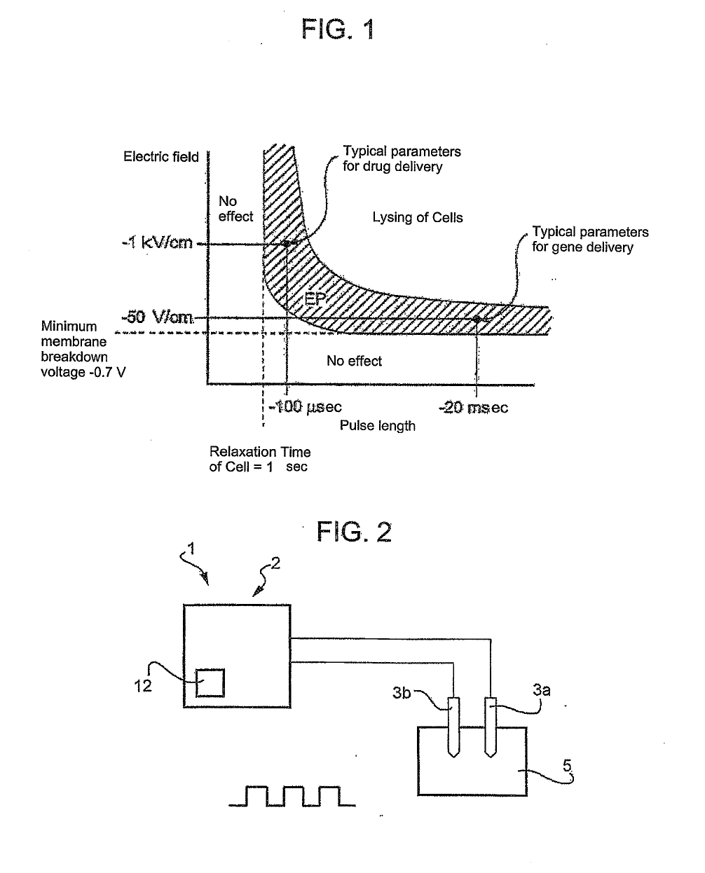

### BEST MODE FOR CARRYING OUT THE INVENTION

In FIG. 2, numeral 1 indicates, as a whole, an electroporation device obtained according to the dictates of the present invention.

Device 1 comprises an electric pulse power generator 2 (of the known type) which is adapted to output a sequence of electric pulses having a predetermined waveform (e.g. square wave) and a constant frequency in the range between 1 and 5000 Hz.

Such electric pulses are delivered to a pair of electrodes 3a, 3b (e.g. a pair of metal needles) introduced in a tissue 5 in which a reversible electroporation process adapted to induce cell death (apoptosis) is to be performed. Tissue 5 may derive from a mammal from which a cell component must be eliminated from the tissue 5.

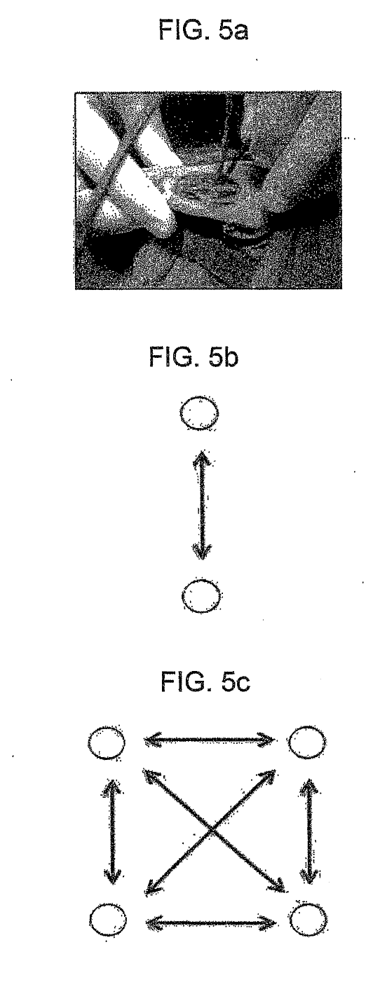

FIG. 2 shows an example in which two electrodes are used; it is clear that the number of electrodes may be higher than two, e.g. a matrix of electrodes may be used (FIG. 5b).

The amplitude (voltage V) of the electric pulses may be adjusted by known techniques; for instance, the maximum amplitude Vmax of the electric pulses may be adjusted in a discrete manner so as to take the following values: 1000 Volt, 1300 Volt, 1500 and 1750 Volt.

In any case, device 1 is configured so as to emit electric pulses, the minimum amplitude Vmin of which allows to generate an electric field which is in any case above the reversible electroporation threshold (250-500-V/cm as a function of the radius of the cells in tissue 5).

Furthermore, the number N of generated pulses is also adjustable by known techniques, in particular the number of generated pulses may vary continuously between a minimum value Nmin (for example 50 pulses) and a maximum number of pulses Nmax (for example 1000 pulses).

According to the present invention, device 1 is provided with a microprocessor control unit 12 that automatically controls the amplitude of the pulses and the number of pulses so as to supply an amount of energy per weight unit (absorbed dose) applied to tissue 5 which is in a range between a first lower limit value of about 3000 J/kg and a second upper limit value of about 4500 J/kg.

Preferably, the range extends between a first lower limit value of about 3500 J/kg and a second upper limit value of about 4500 J/kg.

The optimum value of energy amount per weight unit (optimum absorbed dose) is 4000 J/kg on the basis of the experimental tests carried out.

The combination of pulses and electric field may therefore be described as absorbed dose (J/kg).

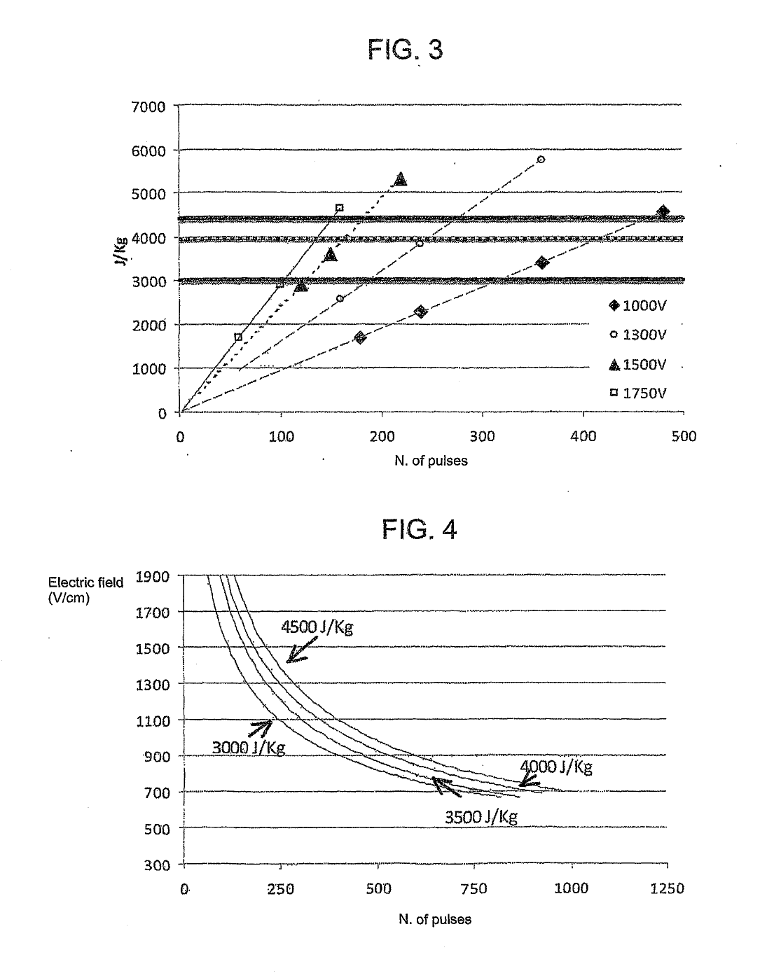

Different combinations of pulses and electric field are effective for cell ablation (see FIG. 3 that shows the number of pulses on the X axis and the value of the absorbed dose (in J/kg) on the Y axis) provided that these combinations provide an amount of energy per weight unit that falls within the above mentioned range (shown in FIG. 3 by means of two horizontal bands). In FIG. 3 the optimum value is shown by a dotted horizontal band.

The following Table 1 shows an example of combinations of amplitude and number of pulses that produce an absorbed dose that falls within the above said range. These combinations have been identified by experimental tests.

| Electric field | | Absorbed |
|---|---|---|
| generated by | | energy |
| the pulses (V/cm) | Number of pulses | (J/kg) |
| 1000 | 320 | 3000 |
| 1000 | 370 | 3500 |
| 1000 | 425 | 4000 |
| 1000 | 480 | 4500 |
| 1300 | 195 | 3000 |
| 1300 | 220 | 3500 |
| 1300 | 255 | 4000 |
| 1300 | 290 | 4500 |
| 1500 | 125 | 3000 |
| 1500 | 150 | 3500 |
| 1500 | 170 | 4000 |
| 1500 | 205 | 4500 |
| 1750 | 100 | 3000 |
| 1750 | 120 | 3500 |
| 1750 | 140 | 4000 |
| 1750 | 160 | 4500 |

Table 1 allows to identify for each value of electric field (1000, 1300, 1500 and 1750 Volts in the example) at least one number of pulses allowing to obtain a value of absorbed dose that falls within the above identified range.

The value of the voltage of the pulses may be manually set by the use of a table of this kind (which may be stored in the memory of the microprocessor of unit 12) so that the number of pulses is automatically determined by device 1 so that the amount of energy per weight unit (absorbed dose) is in any case within the above mentioned range (3000 J/kg-4500 J/kg).

Alternatively, the number of pulses may be manually set so that the pulse voltage value is determined automatically so that the amount of energy per weight unit (absorbed dose) is in the above mentioned range (3000 J/kg-4500 J/kg).

FIG. 4 is a Cartesian diagram that shows the number of pulses required to reach the required absorbed dose value on the y axis as a function of the electric field applied to the electrodes (V/distance in cm) identified on the x axis. Each curve refers to a value of absorbed dose in J/kg (curves associated to values of 3000, 3500, 4000 and 4500 J/kg are shown in the example).

When more than two electrodes are used, a value of energy falling within the above mentioned range is defined for different pairs of electrodes.

The particular above mentioned range of energy amount per weight unit (absorbed dose) of 3000 J/kg to 4500 J/kg results in a reversible electroporation process that implies the destruction of cells according to a different biological mechanism than that resulting from electroporation processes of the known type, e.g. that disclosed in the PCT document cited above.

On the basis of the experiments carried out by the applicant, it appears that the specific above mentioned range of energy amount per weight unit (absorbed dose) contributes to modify the balance of calcium within the cell causing the death of the cell.

In particular, it appears that smaller pores kept open for longer (greater number of pulses) or larger pores maintained for a shorter time (lower number of pulses) determine an alteration of the compartmentalisation of calcium within the cell such as to definitively compromise the function of the mitochondria and of the Golgi apparatus, and therefore produce an irreversible damage to the cell.

The cell death mechanism is therefore different with respect to that of the known art, i.e. not connected to cell lysis or thermal denaturation that implies the use of an electric field having a value such as to cause the destruction of the cell membrane.

Thereby, the ablation of the tissue disclosed herein is carried out by a reversible electroporation that uses lower voltages with respect to the processes involving cell lysis. The electroporation process is therefore less invasive and induces less inflammation.

These results are shown in the experimental tests disclosed hereinafter.

### Example 1

New Zealand white rabbits were used and two or more electrodes were introduced in the distal femur where both bone tissue and cartilage tissue are present. The electrodes were also introduced in the vertebral body of sheep.

The electrodes were introduced in the tissue through an incision in the skin. Stainless steel electrodes were used: two or four electrodes with a diameter of 0.7 mm for rabbits and 2 electrodes with a diameter of 1.2 mm for sheep.

At the end of the electroporation treatment, the electrodes were cut at the bone surface and the portion inside the bone was left where it is, as a marker for later analyses.

Two days after treatment, tetracycline was injected into the rabbits to render fluorescent the new bone laid by osteoblasts.

The experiments were performed in conditions implying the use of different values of electric field (1000, 1300, 1500 and 1750 V/cm) and a number of pulses from 60 to 480; electric pulses lasting 100 microseconds were applied at 4 Hertz. The electrodes were connected in pairs (FIG. 5c). When four electrodes were introduced, all combinations of pairs were used (FIG. 5c). Furthermore, in each individual experimental condition, the same number of pulses between pairs of electrodes was used.

After animals were sacrificed, the bones were removed and cut by means of an Exact 300 band system (Exact Apparatebau GMBH, Nordestedt, Germany). A portion of the sample was decalcified, included in paraffin and coloured with haematoxylin and eosin for histological analysis. A portion of the bone was treated so as to be included in resin without prior decalcification. Briefly, the samples were fixed in 4% formaldehyde for 48 hours, then dehydrated in increasingly concentrated alcohol solutions; after an infiltration time of 24 hours in methyl methacrylate, they were finally included in poly-methylmethacrylate (Merck, Schuchardt, Hohenbrunn, Germany). The blocks of side femur were sectioned along a plane parallel to the diaphyseal axis using a microtome with a Leica 1600 diamond-saw blade (Leica SpA, Milano, Italy) for histology. The emission of tetracyclines by bone neoformation was been observed on unstained sections under fluorescence by means of an optical microscope (Olympus BX41, Melville)(λ=410 nm). As the pores close within a few minutes of the delivery of pulses and the integrity of the functionality of the membrane is recovered, the absence of fluorescence was considered to be the result of the ablation of osteoblasts in that region. The ablated area was quantified by measuring the distance from each electrode surface, at which the bone tissue marked with tetracyclines was observed. Automatic measuring was performed by means of the Q-Win Image Analysis Leica Imaging Systems software (Cambridge, England) on three unstained sections. The sections were later made thinner, polished and finally coloured with toluidine blue and Fast green for the analysis of cell morphology.

### Experiment 1

When a 1000 V/cm electric field was applied, an increasing number of pulses was respectively delivered: 180, 240, 360 and 480. All of the cells were ablated from the tissue when 480 pulses were used.

The following table shows the values of the absorbed dose with respect to the applied electric field and number of pulses.

TABLE 2

| | | Absorbed dose |
|---|---|---|
| V/cm | Number of pulses | (J/kg) |
| 1000 | 180 | 1700 |
| 1000 | 240 | 2271 |
| 1000 | 360 | 3406 |
| 1000 | 480 | 4542 |

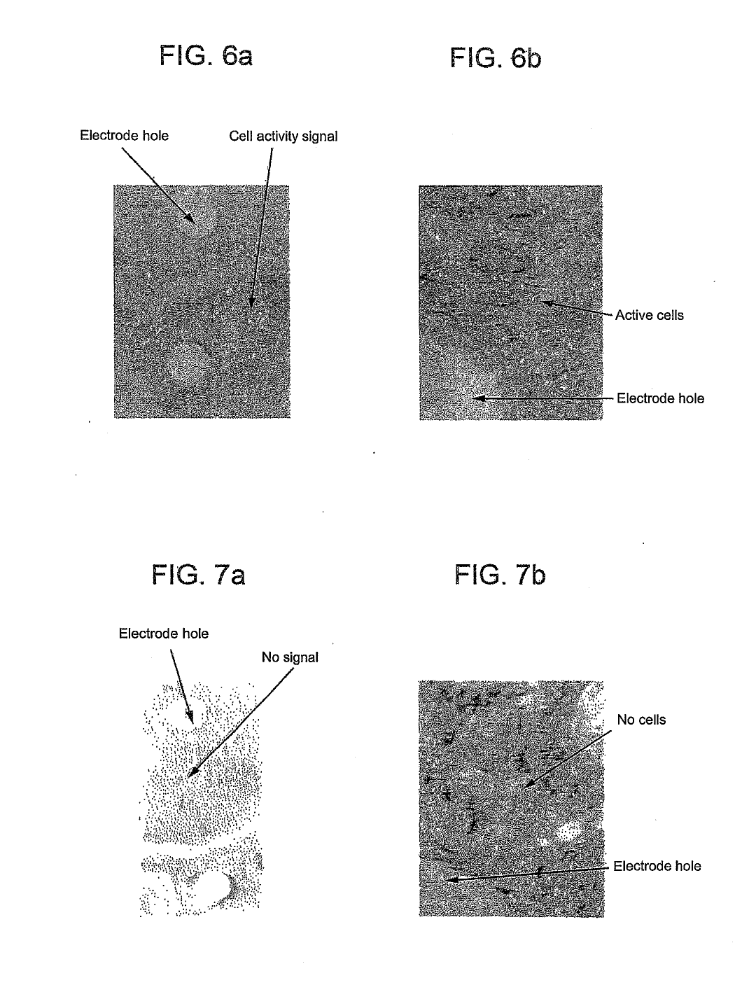

An electroporation at 1000 V/cm, 180 pulses and absorbed dose of 1700 J/kg results in an ineffective ablation. FIG. 6A shows that the fluorescence image highlights the presence of a signal around the electrode. FIG. 6B shows a histological image of a staining with haematoxylin and eosin showing the presence of intact cells.

An electroporation at 1000 V/cm, 480 pulses and absorbed dose of 4542 J/kg instead results in an effective ablation. FIG. 7A shows that the fluorescence image highlights the absence of a signal around the electrode. FIG. 7B shows a histological image of a staining with haematoxylin and eosin showing the complete absence of cells between trabeculae.

### Experiment 2

When a 1300 V/cm electric field was applied, an increasing number of pulses was respectively delivered: 160, 240, 360. All of the cells were ablated from the tissue when 240 and 360 pulses were used.

The following table shows the values of the absorbed dose with respect to the applied electric field and number of pulses.

TABLE 3

| | | Absorbed energy |
|---|---|---|
| V/cm | Number of pulses | (J/kg) |
| 1300 | 160 | 2550 |
| 1300 | 240 | 3833 |
| 1300 | 360 | 5749 |

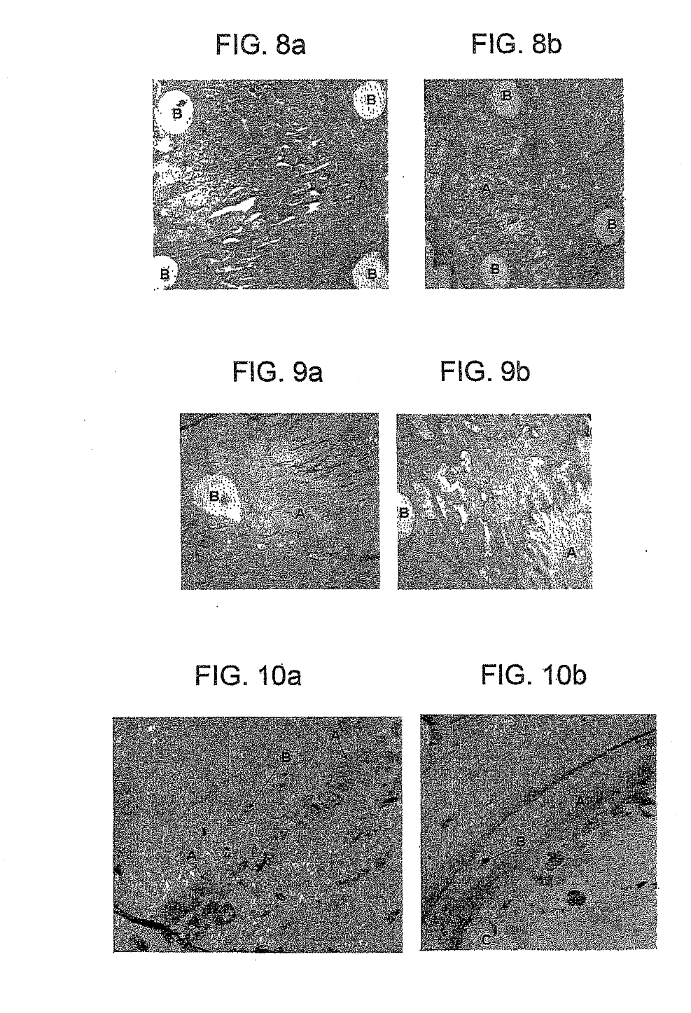

An electroporation at 1300 V/cm, 160 pulses and an absorbed dose of 2550 J/kg of the rabbit distal femur using a four electrode configuration results in an incomplete ablation of the cartilage (A) near the electrodes (B) (FIG. 8A). An electroporation at 1300 V/cm, 360 pulses and an absorbed dose of 5749 J/kg instead results in the complete ablation of the cartilage tissue (A) between the electrodes (B) (FIG. 8B).

### Experiment 3

When a 1500 V/cm electric field was applied to the electrodes, an increasing number of pulses was respectively delivered: 120, 150, 220. All of the cells were ablated from the tissue when 150 and 220 pulses were used.

The following table shows the values of the absorbed dose with respect to the applied electric field and number of pulses.

TABLE 4

| | | Absorbed dose |
|---|---|---|
| V/cm | Number of pulses | (J/kg) |
| 1500 | 120 | 2890 |
| 1500 | 150 | 3598 |
| 1500 | 220 | 5312 |

An electroporation at 1500 V/cm, 120 pulses and an absorbed dose of 2890 J/kg of the rabbit distal femur results in a poor ablation of the cartilage (A) near the electrode (B) (FIG. 9a). An electroporation of 1500 V/cm, 150 pulses and an absorbed dose of 3598 J/kg results instead in the complete ablation of the cartilage tissue (A) near the electrodes (B) (FIG. 9B).

### Experiment 4

When a 1750 V/cm electric field was applied to the electrodes, an increasing number of pulses was respectively delivered: 60, 100, 160. All of the cells were ablated from the tissue when 160 pulses were used.

The following table shows the values of the absorbed dose per applied electric field and number of pulses.

TABLE 5

| | | Absorbed dose |
|---|---|---|
| V/cm | Number of pulses | (J/kg) |
| 1750 | 60 | 1700 |
| 1750 | 100 | 2896 |
| 1750 | 160 | 4633 |

An electroporation of 1750 V/cm, 60 pulses and an absorbed dose of 1700 J/kg of the sheep vertebra results in active osteoblasts (A) on the surface of the trabecula and vital osteocytes (B), which demonstrate an ineffective ablation (FIG. 10A). An electroporation at 1750 V/cm, 160 pulses and an absorbed dose of 4633 J/kg results instead in apoptotic osteoblasts (A) with large cytoplasmic vacuoles and identifiable cell membrane.< The osteocytes (B) are pyknotic. On the surface of the trabecula there are debris of apoptotic cells (C), which demonstrate an effective ablation (FIG. 10B).

1. A reversible electroporation device adapted to induce cell apoptosis, comprising an electric pulse generator adapted to output a sequence of electric pulses having a predetermined waveform delivered to electrodes coupled to a tissue in which a process of reversible electroporation is to be performed,
wherein the device is configured to control the amplitude of the pulses and the number of pulses delivered to the electrodes so as to supply an amount of energy per weight unit (absorbed dose) in a range between a first lower limit value of about 3000 J/kg and a second upper limit value of about 4500 J/kg.

2. The device according to claim 1, wherein the device is configured to control the amplitude of the pulses and the number of pulses delivered to the electrodes so as to supply an amount of energy per weight unit in a range between a first lower limit value of about 3500 J/kg and a second upper limit value of about 4500 J/kg.

3. The device according to claim 1, wherein the device is configured to control the amplitude of the pulses and the number of pulses delivered to the electrodes so as to supply an amount of energy per weight unit in the order of 4000 J/kg.

4. The device according to claim 1, wherein setting means are provided to manually set the amplitude value of the pulses adapted to automatically set the generated number of pulses so that the amount of energy per weight unit falls within said range.

5. The device according to claim 1, wherein setting means are provided to manually set the number of pulses adapted to automatically set the amplitude of pulses generated so that the amount of energy per weight unit falls within said range.

6. A reversible electroporation method adapted to induce cell apoptosis, wherein there is provided the step of delivering a sequence of electric pulses having a predetermined waveform delivered to electrodes coupled with a tissue in which a process of reversible electroporation is to be performed,
comprising the step of controlling the amplitude of the pulses and the number of pulses delivered to the electrodes so as to supply an amount of energy per weight unit (absorbed dose) in a range between a first lower limit value of about 3000 J/kg and a second upper limit value of about 4500 J/kg.

7. The method according to claim 6, wherein:
said amount of energy per weight unit is in the range between a first lower limit value of about 3500 J/kg and a second upper limit value of about 4500 J/kg.

8. The method according to claim 6, wherein said amount of energy per weight unit is in the order of 4000 J/kg.

9. The method according to claim 6, wherein there is provided the step of manually setting the amplitude value of the pulses;
there being provided, in response to said setting step, the automatic generation of a number of pulses such that the amount of energy per weight unit falls within said range.

10. The method according to claim 6, wherein there is provided the step of manually setting the number of pulses;
there being provided, in response to said setting step, the automatic adjustment of the amplitude of the pulses such that the amount of energy per weight unit falls within said range.

11. A method wherein more than two electrodes are used and the minimum value of dose absorbed by the tissue required for complete ablation is calculated for the different pairs of electrodes.
